(19) United States
(12) Patent Application Publication
(10) Pub. No.: US 2008/0249556 A1
(43) Pub. Date: Oct. 9, 2008

(54) DISSECTION APPARATUS AND DISSECTION METHOD

(76) Inventor: Ken YAMATANI, Koganei-shi (JP)

Correspondence Address: OSTROLENK FABER GERB & SOFFEN, 1180 AVENUE OF THE AMERICAS, NEW YORK, NY, 100368403, US

(21) Appl. No.: 11/697,502

(22) Filed: Apr. 6, 2007

(51) Int. Cl. A61B 17/00

(52) U.S. Cl. 606/190

(57) ABSTRACT

A dissection apparatus includes a main body portion which extends from a proximal end side to a distal end side, and a dissecting portion which is provided to a distal end of the main body portion and adapted to dissect a blood vessel from a living tissue, the dissecting portion including a flat portion which is to be pressed against the blood vessel.

### BACKGROUND OF THE INVENTION

1. Field of the Invention

The present invention relates to a dissection apparatus and dissection method of a blood vessel from a living tissue.

2. Description of the Related Art

Jpn. Pat. Appln. KOKAI Publication No. 9-75354 discloses a dissection apparatus to dissect a blood vessel from a blood vessel connective tissue in order to sample a subcutaneous blood vessel such as a saphenous vein of a lower limb under endoscopic observation. In the dissection apparatus, a distal end portion for dissection is disposed at the distal end of a cylindrical main body portion. The distal end portion includes a tapered conical shape as a whole so it can be easily inserted between the blood vessel connective tissue and the blood vessel, and its distal end forms a locally blunt shape so it will not apply any unwanted force to the blood vessel.

### BRIEF SUMMARY OF THE INVENTION

In an aspect of the present invention, a dissection apparatus is characterized by including: a main body portion which extends from a proximal end side to a distal end side; and a dissecting portion which is provided to a distal end of the main body portion and adapted to dissect a blood vessel from a living tissue, the dissecting portion including a flat portion which is to be pressed against the blood vessel.

In another aspect of the present invention, a dissection method of a blood vessel using a dissection apparatus including a dissecting portion with a flat portion, characterized by including: moving the dissecting portion forward in a running direction of a main duct, while pressing the flat portion against the main duct, to dissect the main duct from a living tissue; moving the dissecting portion forward in a direction across a running direction of a branch, while pressing the flat portion against the branch, to dissect the branch from the living tissue; and inserting the dissecting portion in an undissected tissue near the branch, and pivoting the dissecting portion around the direction across the running direction of the branch to dissect the branch from the undissected tissue.

In another aspect of the present invention, a dissection apparatus is characterized by including: a main body portion which extends from a proximal end side to a distal end side; and, a dissecting portion which is provided to a distal end of the main body portion and adapted to dissect a blood vessel from a living tissue, the dissecting portion including a transparent portion to observe an outside of the dissecting portion from inside thereof by an endoscope, and the transparent portion including a mark which is observable by the endoscope.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a view showing a dissection system according to the first embodiment of the present invention;

FIG. 2A is a perspective view showing a dissecting portion according to the first embodiment of the present invention;

FIG. 2B is a top view showing the dissecting portion according to the first embodiment of the present invention;

FIG. 2C is a side view showing the dissecting portion according to the first embodiment of the present invention;

FIG. 3A is a top view showing a dissecting portion according to a modification of the first embodiment of the present invention;

FIG. 3B is a side view showing the dissecting portion according to the modification of the first embodiment of the present invention;

FIG. 4A is a schematic view showing a saphenous vein of a lower limb which is to be dissected by a dissection method according to the first embodiment of the present invention;

FIG. 4B is a schematic view showing a cutaneous vein of an upper limb which is to be dissected by the dissection method according to the first embodiment of the present invention;

FIG. 5 is a schematic view to explain the dissection method according to the first embodiment of the present invention;

FIG. 6A is a view showing an endoscopic image in a step of dissecting a main duct in the dissection method according to the first embodiment of the present invention;

FIG. 6B is a view showing an endoscopic image in a stage before a step of dissecting a branch in the dissection method according to the first embodiment of the present invention;

FIG. 6C is a view showing an endoscopic image in a step of dissecting the branch in the dissection method according to the first embodiment of the present invention;

FIG. 6D is a view showing an endoscopic image in a step of dissecting from an undissected tissue in the dissection method according to the first embodiment of the present invention;

FIG. 7A is a perspective view showing a dissecting portion according to the second embodiment of the present invention;

FIG. 7B is a top view showing the dissecting portion according to the second embodiment of the present invention;

FIG. 7C is a side view showing the dissecting portion according to the second embodiment of the present invention;

FIG. 8A is a top view showing a dissecting portion according to a modification of the second embodiment of the present invention;

FIG. 8B is a side view showing the dissecting portion according to the modification of the second embodiment of the present invention;

FIG. 9 is a view showing an endoscopic image in a dissection method according to the second embodiment of the present invention;

FIG. 10A is a perspective view showing a dissecting portion according to a third embodiment of the present invention;

FIG. 10B is a top view showing the dissecting portion according to the third embodiment of the present invention;

FIG. 10C is a side view showing the dissecting portion according to the third embodiment of the present invention;

FIG. 11 is a top view showing a dissecting portion according to the fourth embodiment of the present invention;

FIG. 12A is a perspective view showing a dissecting portion according to a fifth embodiment of the present invention;

FIG. 12B is a top view showing the dissecting portion according to the fifth embodiment of the present invention;

FIG. 12C is a side view showing the dissecting portion according to the fifth embodiment of the present invention; and

FIG. 13 is a schematic view to explain a dissection method according to the fifth embodiment of the present invention.

### DETAILED DESCRIPTION OF THE INVENTION

The respective embodiments of the present invention will be described hereinafter with reference to the accompanying drawing.

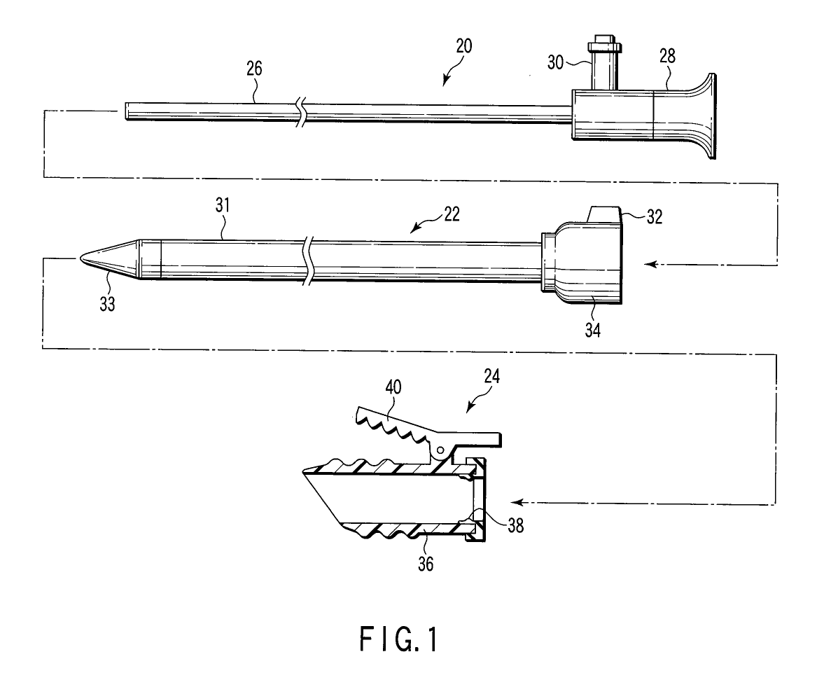

FIGS. 1 to 6D show the first embodiment of the present invention and its modification.

Referring to FIG. 1, a dissection system according to the embodiment includes an endoscope 20 to perform observation in a body, a dissection apparatus 22 to dissect a blood vessel in the body, and a trocar 24 to insert the endoscope 20 and dissection apparatus 22 into the body.

The endoscope 20 is a rigid endoscope and includes an elongated rod-like inserting portion 26. The proximal end of the inserting portion 26 connects to an eyepiece 28 to observe an endoscopic image. A lightguide connecter 30 projects from the eyepiece 28. The lightguide connecter 30 connects to a lightguide cable to supply illumination light to the endoscope 20.

The dissection apparatus 22 includes a tubular main body portion 31 where the endoscope 20 is to be inserted to be movable forward/backward. A connecting portion 34 to connect to the eyepiece 28 of the endoscope 20 is disposed on the proximal end of the main body portion 31. More specifically, the connecting portion 34 includes a notch 32 where the lightguide connecter 30 of the endoscope 20 is to be inserted and engaged. A dissecting portion 33 to dissect a blood vessel is disposed at the distal end of the main body portion 31. The dissecting portion 33 will be described later in detail.

The trocar 24 includes a guide pipe 36 to guide the dissection apparatus 22 into the body. A seal 38 is disposed on the inner surface of the proximal end of the guide pipe 36. The seal 38 allows the dissection apparatus 22 to be inserted in the guide pipe 36 airtightly and to be movable forward/backward. The outer surface of the guide pipe 36 includes a projection to engage with the living tissue. The trocar 24 also includes a holding portion 40 to hold the guide pipe 36 onto the living tissue.

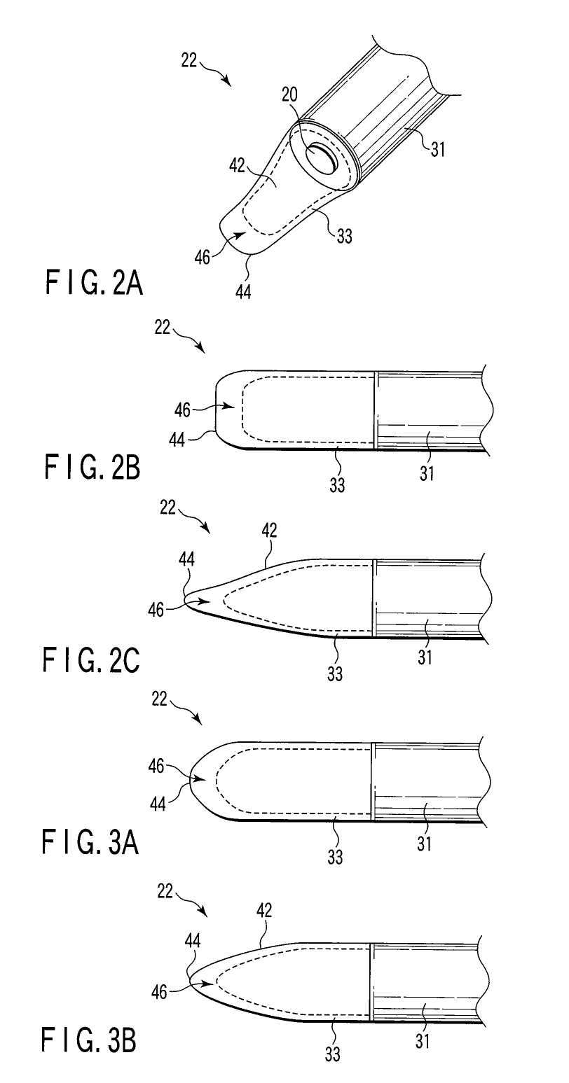

The dissecting portion 33 will be described in detail with reference to FIGS. 2A to 2C.

The dissecting portion 33 according to the embodiment forms a wide shape extending from the proximal end side to the distal end side while keeping almost the same width when seen in one direction perpendicular to the longitudinal direction of the main body portion 31 (to be referred to as the vertical direction hereinafter), and a tapered shape when seen in a direction perpendicular to the longitudinal direction of the main body portion 31 and one direction described above (to be referred to as the right-and-left direction hereinafter). In other words, the entire dissecting portion 33 forms a diameter decreasing portion with a diameter that decreases from the proximal end side to the distal end side. The upper surface of the dissecting portion 33 includes a rectangular flat portion 42 which extends from the distal end toward the proximal end side of the dissecting portion 33 while keeping almost the same width. The flat portion 42 is to be pressed against a blood vessel.

The dissecting portion 33 is formed of a hollow transparent member including an almost constant thickness. The entire dissecting portion 33 forms a transparent portion. By the endoscope 20 which is inserted in the main body portion 31 and projects from the distal end of the main body portion 31, observation of the outside from the inside of the dissecting portion 33 is enabled. At the distal end of the dissecting portion 33, an acute-angled shape 44 which forms an acute angle when seen in the right-and-left direction extends in the direction of width when seen in the vertical direction. The distal end of the acute-angled shape 44 locally forms a blunt shape. The inner surface of the dissecting portion 33 includes a shape that corresponds to its outer surface. Bend line portions on the outer surface and the inner surface of the acute-angled shape 44 form a linear mark 46 which can be observed by the endoscope 20. The mark 46 is to be arranged at almost the center of the field of view of the endoscope 20.

FIGS. 3A and 3B show a modification of the embodiment.

In the dissecting portion 33 according to this modification, when seen in the vertical direction, at its distal end side, the width decreases from the proximal end side toward the distal end side almost axi-symmetrically about the center axis. Note that when seen in the vertical direction, the distal end side of the dissecting portion 33 does not converge to a vertex. At the distal end of the dissecting portion 33, the acute-angled shape 44 similar to that of the first embodiment extends in the direction of width. The dissecting portion 33 of this modification also forms the linear mark 46.

A method for using the dissection apparatus 22 according to the embodiment will be described.

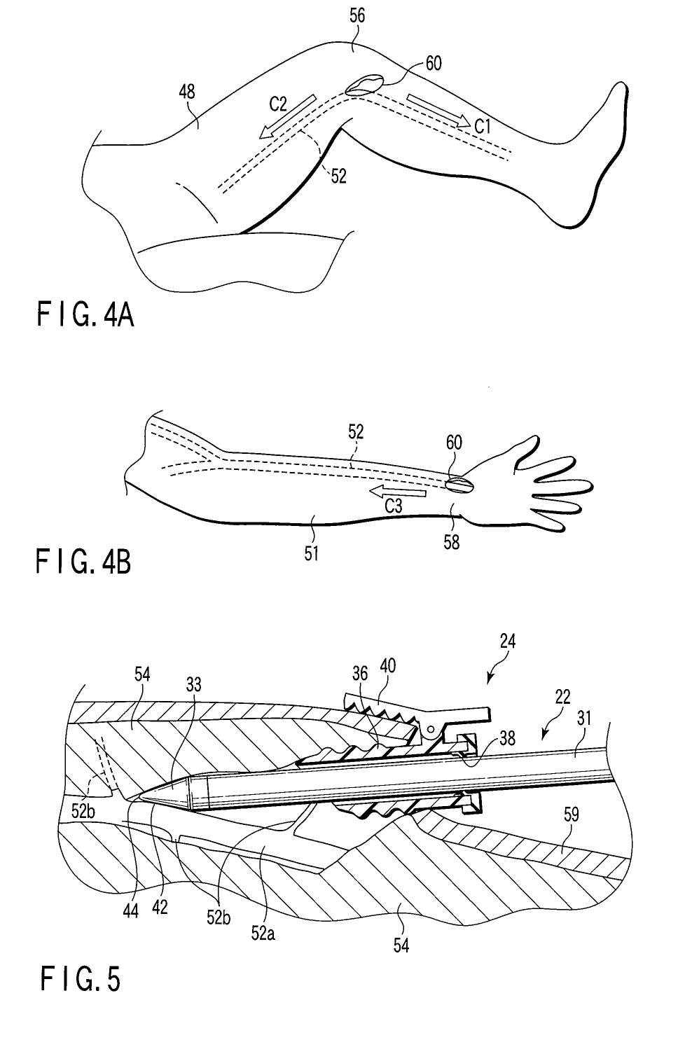

Referring to FIGS. 4A, 4B, and 5, an operation of sampling a blood vessel 52 such as a saphenous vein of a lower limb 48 or a cutaneous vein of an upper limb 51 is performed to use the sampled blood vessel 52 in coronary artery bypass surgery. When sampling the blood vessel 52, the blood vessel 52 must be dissected from a blood vessel connective tissue 54. In the following description, a dissection method of the blood vessel 52 will be described in detail in respective steps.

Preparation Step

Referring to FIGS. 4A, 4B, and 5, in a knee 56 or wrist 58, a skin 59 is incised immediately above the blood vessel 52 to be sampled, to form an incision 60. The guide pipe 36 of the trocar 24 is inserted in the incision 60 and held by the holding portion 40 with respect to the incision 60. The endoscope 20 is inserted in the dissection apparatus 22. The lightguide connecter 30 of the endoscope 20 is inserted in and engaged with the notch 32 of the dissection apparatus 22, to lock the dissection apparatus 22 and endoscope 20 with each other. The distal end of the endoscope 20 is caused to project from the distal end of the main body portion 31 of the dissection apparatus 22 and arranged within the dissecting portion 33. Subsequently, the endoscope 20 and dissection apparatus 22 are inserted into the body through the trocar 24. During this insertion, due to the seal 38 of the trocar 24, the dissection apparatus 22 is inserted in the guide pipe 36 airtightly and to be movable forward/backward.

Step of Dissecting Main Duct

As indicated by arrows C1, C2, and C3 in FIGS. 4A and 4B, the dissection apparatus 22 inserted in the body dissects a main duct 52a of the blood vessel 52 from the blood vessel connective tissue 54.

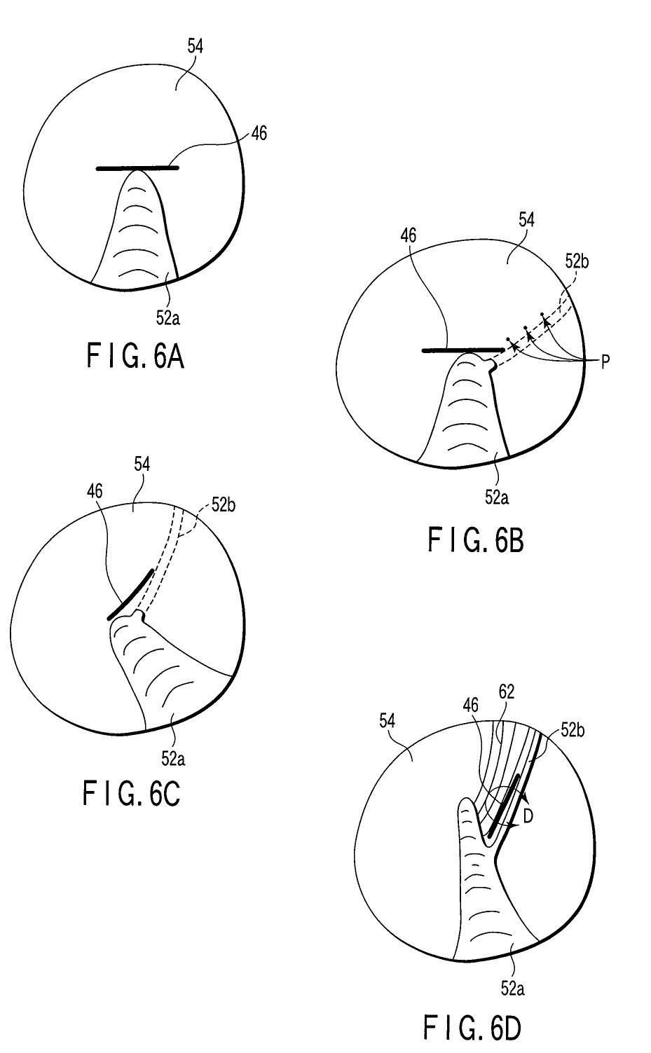

More specifically, as shown in FIG. 6A, in the observation image of the endoscope 20, the linear mark 46 is arranged at almost the center of the field of view of the endoscope 20. The mark 46 indicates the acute-angled shape 44 of the dissecting portion 33. The relative positional relationship between the acute-angled shape 44 and main duct 52a can be understood from the relative positional relationship between the mark 46 and main duct 52a. By manipulating the dissection apparatus 22, the dissecting portion 33 is arranged such that its flat portion 42 faces the main duct 52a and the main duct 52a extends from the peripheral portion to the central portion in the observation image of the endoscope 20. While keeping the mark 46 in constant contact with the outer surface of the main duct 52a in the observation image of the endoscope 20, the dissection apparatus 22 is pushed in. Consequently, with the flat portion 42 being pressed against the main duct 52a, the dissecting portion 33 is moved forward along the main duct 52a, and the acute-angled shape 44 at the distal end of the dissecting portion 33 is inserted between the blood vessel connective tissue 54 and the main duct 52a to dissect the main duct 52a from the blood vessel connective tissue 54.

Step of Dissecting Branch Subsequently, a branch 52b branching from the main duct 52a is dissected from the blood vessel connective tissue 54.

More specifically, first, as shown in FIG. 6B, the branch 52b is detected in the observation image of the endoscope 20.

If the dissecting portion includes a conical shape, its distal end is moved across the branch 52b and inserted between the blood vessel connective tissue 54 and the branch 52b. By repeating this insertion at a large number of positions P along the running direction of the branch 52b, the branch 52b is dissected.

In contrast to this, with the dissection apparatus 22 according to the embodiment, as shown in FIG. 6C, the dissection apparatus 22 is manipulated to arrange the dissecting portion 33 such that the flat portion 42 of the dissecting portion 33 faces the branch 52b, and that the mark 46 is located at the boundary portion of the blood vessel connective tissue 54 and the branch 52b to be almost parallel to the running direction of the branch 52b in the observation image of the endoscope 20. The dissection apparatus 22 is then pushed in. Consequently, the acute-angled shape 44 at the distal end of the dissecting portion 33 is inserted between the blood vessel connective tissue 54 and the branch 52b. With the flat portion 42 being pressed against the branch 52b, the dissecting portion 33 is moved forward across the branch 52b, to dissect the branch 52b from the blood vessel connective tissue 54.

Step of Dissecting from Undissected Tissue

As shown in FIG. 6D, an undissected tissue 62 such as a funicular tissue may be left near the branch 52b. In this case, in the same manner as in the step of dissecting the branch, the dissection apparatus 22 is manipulated to arrange the dissecting portion 33 such that its flat portion 42 faces the branch 52b, and that the mark 46 is located at a boundary portion of the branch 52b and the undissected tissue 62 to be almost parallel to the running direction of the branch 52b in the observation image of the endoscope 20. The dissection apparatus 22 is then pushed in. Consequently, the acute-angled shape 44 at the distal end of the dissecting portion 33 is inserted between the undissected tissue 62 and branch 52b. With the dissecting portion 33 being inserted between the undissected tissue 62 and branch 52b, the dissection apparatus 22 is rotated about its central axis. The dissecting portion 33 is pivoted while pressing the flat portion 42 against the branch 52b, as indicated by an arrow D in FIG. 6D, to dissect the branch 52b from the undissected tissue 62.

Where necessary, in the step of dissecting the main duct, with the dissecting portion 33 being inserted between the blood vessel connective tissue 54 and the main duct 52a, the dissection apparatus 22 may be rotated about its central axis and dissecting portion 33 may be pivoted with the pressing portion of the main duct 52a and the flat portion 42 as a fulcrum, to dissect the main duct 52a from the blood vessel connective tissue 54.

Similarly, in the step of dissecting the branch, with the dissecting portion 33 being inserted between the blood vessel connective tissue 54 and the branch 52b, the dissection apparatus 22 may be rotated about its central axis and the dissecting portion 33 may be pivoted while pressing the flat portion 42 against the branch 52b, to dissect the branch 52b from the blood vessel connective tissue 54.

Therefore, the dissection apparatus 22 according to the embodiment includes the following effects.

In the dissection apparatus 22 according to the embodiment, in the dissecting operation, the dissecting portion 33 rarely disengages from the main duct 52a or branch 52b and the main duct 52a and branch 52b can be dissected by the pivotal motion of the dissecting portion 33 in addition to the forward movement. This enables to perform dissection of the main duct 52a and branch 52b easily and reliably. In particular, as the dissecting portion 33 that forms the diameter decreasing portion is provided with the flat portion 42, when inserting the dissecting portion 33 between the blood vessel connective tissue 54 and the main duct 52a or branch 52b, the dissecting portion 33 rarely disengages from the main duct 52a or branch 52b and by pivoting the dissecting portion 33 inserted between the blood vessel connective tissue 54 and the main duct 52a or branch 52b, the main duct 52a or branch 52b can be dissected easily. As the flat portion 42 extends from the distal end of the dissecting portion 33, the distal end of the dissecting portion 33 rarely disengages from the main duct 52a or branch 52b. This sufficiently prevents the entire dissecting portion 33 from disengaging from the main duct 52a or blood vessel 52.

The relative positional relationship between the main duct 52a or branch 52b and the dissecting portion 33 can be understood from the relative positional relationship between the main duct 52a or branch 52b and the mark 46 in the field of view of the endoscope 20. In particular, the acute-angled shape 44 to be inserted in the boundary region of the blood vessel connective tissue 54 and the main duct 52a or branch 52b forms the mark 46, the mark 46 is arranged at the center of the field of view of the endoscope 20, and the boundary region as the dissection target portion is also arranged at the center of the field of view of the endoscope 20. Thus, the relative positional relationship between the boundary region as the dissection target portion and the acute-angled shape 44 can be understood easily. As the mark 46 is linear and extends in a direction, the direction and posture of the dissecting portion 33 can be understood easily. Hence, dissection of the main duct 52a or branch 52b can be performed easily and reliably.

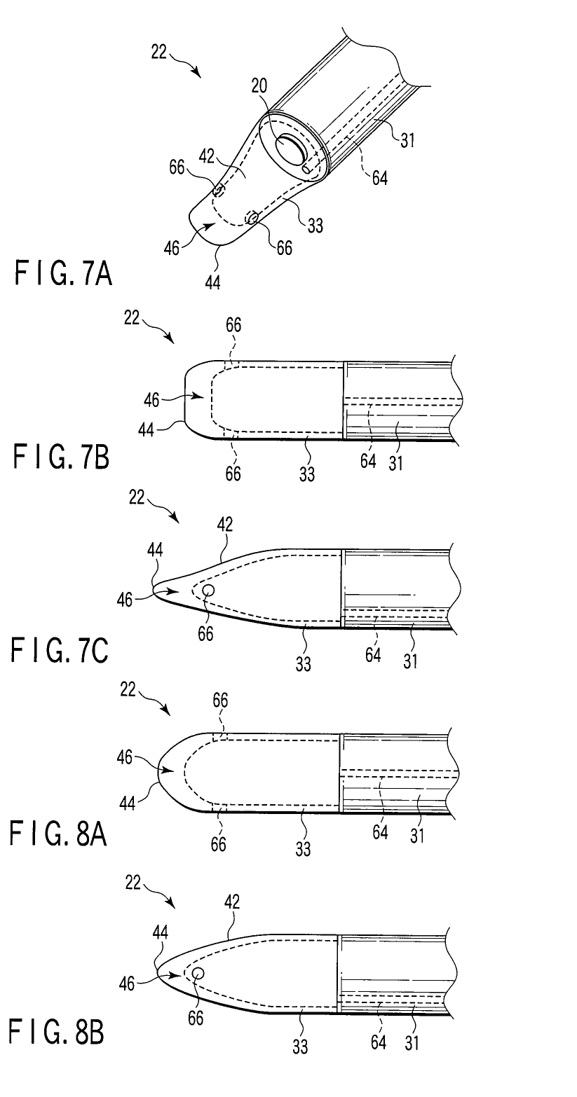

FIGS. 7A to 9 show the second embodiment of the present invention and its modification.

In a dissection apparatus 22 according to the embodiment, an air supply path 64 extends in its main body portion 31. The proximal end of the air supply path 64 communicates with an air supply connecter projecting from the proximal end of the main body portion 31. The distal end of the air supply path 64 opens to the interior of the dissecting portion 33 at the distal end of the main body portion 31. At the distal end of the main body portion 31, the endoscope 20 is airtightly projected/retracted. The distal end of the dissecting portion 33 includes air supply holes 66 through which the inside and outside of the dissecting portion 33 communicate with each other. The air supply holes 66 are arranged in the dissecting portion 33, which forms the diameter decreasing portion, on the two sides of the flat portion 42 when seen in the vertical direction, to be held within the field of view of the endoscope 20, and open to the right-and-left direction.

FIGS. 8A and 8B show the modification of the embodiment.

The dissecting portion 33 of the embodiment includes the same shape as that of the dissecting portion 33 of the modification of the first embodiment, and the air supply hole 66 similar to each of those of the second embodiment.

A method for using the dissection apparatus 22 according to the embodiment will be described.

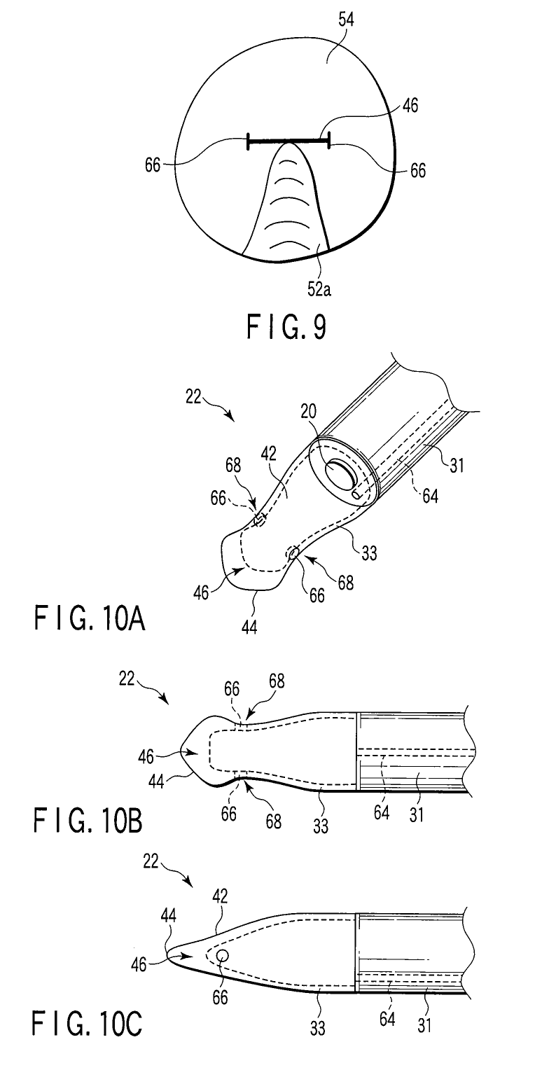

The dissecting operation of the blood vessel 52 by the dissection apparatus 22 according to the embodiment is the same as that by the dissection apparatus 22 according to the first embodiment. Note that air is supplied from the air supply holes 66 through the air supply connecter, air supply path 64, and the interior of dissecting portion 33 to enlarge the inner cavity where the dissecting operation is to be performed. By observing the states of the blood vessel connective tissue 54 and the main duct 52a or the branch 52b near the air supply hole 66 by the endoscope 20, as shown in FIG. 9, the air supply state from the air supply holes 66 can be understood.

Therefore, the dissection apparatus 22 according to the embodiment includes the following effects.

In the dissection apparatus 22 according to the embodiment, air supplied from the air supply holes 66 enlarges the inner cavity. In particular, the air supply holes 66 are formed at the dissecting portion 33 which forms the diameter decreasing portion to be inserted between the blood vessel connective tissue 54 and the main duct 52a or branch 52b. This enables to enlarge the portion of the inner cavity, which tend to be clogged and affect observation and dissecting operation. The air supply holes 66 are arranged, of the dissecting portion 33, neither on the flat portion 42 nor on the side that opposes the flat portion 42 that can be easily covered by the main duct 52a, the branch 52b, or the blood vessel connective tissue 54, but on the two sides of the flat portion 42. This prevents the air supply holes 66 from being clogged to interfere with air supply. Hence, observation of the interior of the inner cavity by the endoscope 20 and the dissecting operation itself becomes easy, so dissection of the main duct 52a or branch 52b can be performed sufficiently easily and reliably.

FIGS. 10A to 10C show the third embodiment of the present invention.

According to the embodiment, the width of the dissecting portion 33 decreases once, and then increases and decreases again from the proximal end side toward the distal end side when seen in the vertical direction, so the distal end portion of the dissecting portion 33 converges. Therefore, recessed shapes 68 are formed on the two sides of the dissecting portion 33 when seen in the vertical direction. The air supply holes 66 similar to those of the second embodiment are arranged at the bottoms of the respective recessed shapes 68. The dissecting operation by a dissection apparatus 22 of the embodiment is the same as that by the dissection apparatus 22 of the second embodiment. In the dissection apparatus 22 of the embodiment, the air supply holes 66 are formed in the bottoms of the recessed shapes 68. This reliably prevents the air supply holes 66 from being clogged by the blood vessel connective tissue 54, the main duct 52a, or the branch 52b. Also, a space is reserved in front of the openings of the air supply holes 66. Thus, air supply can be always performed smoothly.

FIG. 11 shows the fourth embodiment of the present invention.

The dissecting portion 33 according to the embodiment includes almost the same shape as that of the dissecting portion 33 of the third embodiment. Note that the openings of air supply holes 66 face obliquely backward when seen in the vertical direction. When inserting the dissecting portion 33 between the blood vessel connective tissue 54 and the main duct 52a or the branch 52b, the more forward the air supply holes 66 are directed, the more the blood vessel connective tissue 54, the main duct 52a or the branch 52b tend to clog the air supply holes 66. As the air supply holes 66 of the embodiment face obliquely backward, clogging of the air supply holes 66 is prevented reliably.

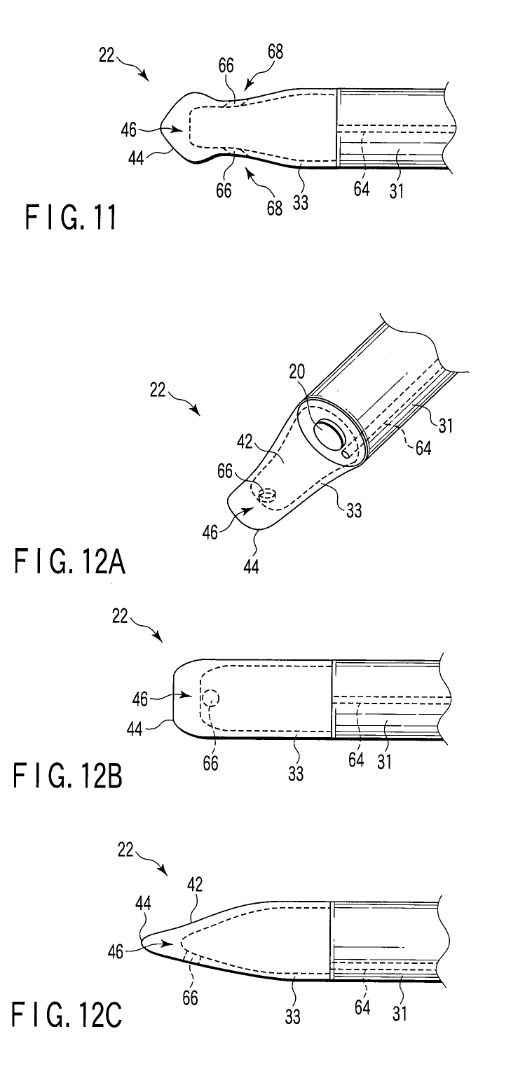

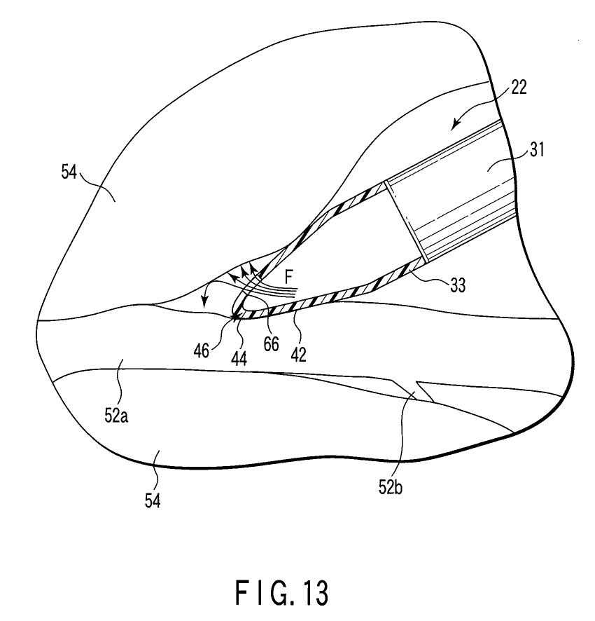

FIGS. 12A to 13 show the fifth embodiment of the present invention.

The dissecting portion 33 according to the embodiment includes the same shape as that of the dissecting portion 33 of the first embodiment. The distal end of the dissecting portion 33 includes the air supply hole 66 on the side that opposes the flat portion 42. The dissecting operation by the dissection apparatus 22 according to the embodiment is the same as that by the dissection apparatus 22 according to the second embodiment. Air supply from the air supply hole 66 enlarges the inner cavity. Air supplied from the air supply hole 66 is introduced to the space in the vicinity of the dissection target portion between the blood vessel connective tissue 54 and the main duct 52a, as indicated by arrows F. The pressure of the supplied air serves to dissect the main duct 52a from the blood vessel connective tissue 54. Hence, dissection of the main duct 52a is promoted by air supply.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A dissection apparatus comprising:
a main body portion which extends from a proximal end side to a distal end side; and
a dissecting portion which is provided to a distal end of the main body portion and adapted to dissect a blood vessel from a living tissue,
the dissecting portion including a flat portion which is to be pressed against the blood vessel.

2. The dissection apparatus according to claim 1, wherein
the dissecting portion includes a diameter decreasing portion a diameter of which decreases from the proximal end side to the distal end side, and
the flat portion is provided to the diameter decreasing portion.

3. The dissection apparatus according to claim 1, wherein the flat portion extends from a distal end of the dissecting portion to the proximal end side thereof.

4. The dissection apparatus according to claim 1, wherein the dissecting portion includes an air supply hole to supply gas from inside of the dissecting portion to outside thereof.

5. The dissection apparatus according to claim 4, wherein the dissection apparatus is used together with an endoscope,
and the air supply hole is adapted to be arranged within a field of view of the endoscope.

6. The dissection apparatus according to claim 2, wherein the dissecting portion includes an air supply hole which is formed in the diameter decreasing portion to supply gas from inside of the dissecting portion to outside thereof.

7. The dissection apparatus according to claim 4, wherein the air supply hole is arranged on a side of the flat portion in the dissecting portion.

8. The dissection apparatus according to claim 4, wherein the air supply hole is arranged to oppose the flat portion in the dissecting portion.

9. A dissection method of a blood vessel using a dissection apparatus including a dissecting portion with a flat portion, comprising:
moving the dissecting portion forward in a running direction of a main duct, while pressing the flat portion against the main duct, to dissect the main duct from a living tissue;
moving the dissecting portion forward in a direction across a running direction of a branch, while pressing the flat portion against the branch, to dissect the branch from the living tissue; and
inserting the dissecting portion in an undissected tissue near the branch, and pivoting the dissecting portion around the direction across the running direction of the branch to dissect the branch from the undissected tissue.

10. A dissection apparatus comprising:
a main body portion which extends from a proximal end side to a distal end side; and
a dissecting portion which is provided to a distal end of the main body portion and adapted to dissect a blood vessel from a living tissue,
the dissecting portion including a transparent portion to observe an outside of the dissecting portion from inside thereof by an endoscope, and
the transparent portion including a mark which is observable by the endoscope.

11. The dissection apparatus according to claim 10, wherein the mark is arranged at a center of a field of view of the endoscope.

12. The dissection apparatus according to claim 10, wherein the mark is linear.

13. The dissection apparatus according to claim 10, wherein the mark is formed of an acute-angled shape of the transparent portion.
